(19) United States
(12) Patent Application Publication
(10) Pub. No.: US 2026/0155594 A1
(43) Pub. Date: Jun. 4, 2026

(54) BOARD-END CONNECTOR AND BIOSENSOR DEVICE COMPRISING THE SAME

(71) Applicant: SUYIN CORPORATION, New Taipei City (TW)

(72) Inventors: Guang Jin MENG, New Taipei City (TW); Meng Li CHENG, New Taipei City (TW)

(21) Appl. No.: 19/332,175

(22) Filed: Sep. 18, 2025

(30) Foreign Application Priority Data
Dec. 3, 2024 (CN) 2024117561238

(51) Int. Cl. H01R 12/72; A61B 5/145; A61B 5/1486

(52) U.S. Cl. CPC H01R 12/722; A61B 5/14532; A61B 5/14865; A61B 2562/166; A61B 2562/227

(57) ABSTRACT

A board-end connector and a biosensor device comprising the same are disclosed. The board-end connector includes an insulator having a recess and a plurality of conductive members fixed to the insulator. Each conductive member includes a first elastic arm and a second elastic arm that are electrically connected to each other and correspondingly contact two electronic devices. A first electrical contact portion disposed at an upper end of the first elastic arm is exposed at an upper portion of the insulator. The recess is configured to accommodate one electronic device. A second electrical contact portion disposed at an upper end of the second elastic arm is exposed within the recess.

### CROSS REFERENCE TO RELATED APPLICATIONS

This application claims priority to China Application Serial No. 2024117561238, filed Dec. 3, 2024, the disclosure of which is incorporated herein by reference.

### FIELD OF DISCLOSURE

The present application relates to the field of connector technology, and more particularly to a board-end connector and a biosensor device comprising the same.

### BACKGROUND

With the advancement of technology, various user-friendly medical diagnostic devices have been continuously developed and manufactured. A glucometer is one such device configured to measure the blood glucose level of a human body. The glucometer typically operates based on an electrochemical method, in which a current signal generated during a reaction process is used to determine the glucose concentration. Electrons generated from a reaction between an enzyme and glucose are counted by a current measurement apparatus and converted into a glucose concentration value.

A compact and portable blood glucose monitoring device, also referred to as a continuous glucose monitor, has become commercially available. The continuous glucose monitor is wearable and capable of continuously collecting glucose data from the human body, features a small form factor, and causes minimal pain, thereby being widely adopted in glucose monitoring for diabetic patients. The continuous glucose monitor includes a main unit and an implantable sensor. The implantable sensor is implanted subcutaneously and converts the blood glucose levels into electrical signals. The main unit processes, analyzes, stores, and outputs the electrical signals generated by the sensor, and transmits the data to a mobile application. The mobile application displays related information, such as real-time glucose values and glucose variation trends.

As shown in FIG. 1, a portable blood glucose monitoring device 100 includes a housing 103, a sensor 101, and a PCB (Printed Circuit Board, not shown). The PCB is mounted inside the housing 103. The sensor 101 is positioned in a groove 104 formed in the housing 103. Due to the compact size of the portable blood glucose monitoring device 100, an internal space of the housing 103 is limited. Electrical connections between the sensor 101 and the PCB are typically achieved by serially connecting multiple semiconductor components 102 to the sensor 101, followed by connecting the semiconductor components 102 to the PCB. The small size of the semiconductor components 102 and the need for multiple assembly steps to mount the semiconductor components 102 onto the PCB result in assembly inefficiency. In addition, establishing electrical connection between the sensor 101 and the semiconductor components 102 in series typically requires soldering, but the limited internal space makes the soldering process difficult.

In view of the foregoing, the present application provides the following technical solution.

### SUMMARY OF DISCLOSURE

The present application provides board-end connector and a biosensor device including the same to address the issues in the prior art.

To address the above technical problem, in a first aspect the present application provides a board-end connector. The board-end connector includes an insulator and a plurality of conductive members.

The insulator includes a recess. The plurality of conductive members are fixed on the insulator, and each of the plurality of conductive members includes a first elastic arm and a second elastic arm. The first elastic arm includes a first electrical contact portion. The first electrical contact portion is disposed at an upper end of the first elastic arm and exposed at an upper portion of the insulator. The second elastic arm includes a second electrical contact portion. The second electrical contact portion is disposed at an upper end of the second elastic arm and exposed in the recess.

The first elastic arm and the second elastic arm are electrically connected to each other and are correspondingly configured to electrically contact two electronic devices. The recess is configured to accommodate one of the two electronic devices.

Optionally, in one embodiment of the present application, each of the plurality of conductive members further includes a main body portion, the first elastic arm and the second elastic arm are connected via the main body portion, and a top surface of the first electrical contact portion is higher than a top surface of the second electrical contact portion.

Optionally, in one embodiment of the present application, each of the plurality of conductive members includes two second elastic arms arranged in parallel and a bending portion. Root portions of the two second elastic arms are connected to the bending portion. The bending portion is integrally connected to the main body portion.

Optionally, in one embodiment of the present application, the insulator is integrally formed with and fixed to the plurality of conductive members by an insert molding process.

Optionally, in one embodiment of the present application, the plurality of conductive members are fixed to the insulator by an insertion. The insulator defines at least one first window, at least one second window and at least one positioning hole. The first window and the second window are spaced apart from each other, such that the second window is configured to extend through the recess.

Each of the plurality of conductive members further includes at least one positioning tab. Each of the plurality of conductive members is assembled to the insulator by fitting the positioning tab into the positioning hole. The first electrical contact portion disposed at the upper end of the first elastic arm and the second electrical contact portion disposed at the upper end of the second elastic arm are configured to correspondingly extend through the first window and the second window.

Optionally, in one embodiment of the present application, the positioning tab includes an arcuate locking portion and a protruding part. The arcuate locking portion is formed extending along a width direction on both sides of an end portion of the positioning tab. The protruding part is formed by a stamping process at the end portion of the positioning tab and projects along a thickness direction of the positioning tab. The protruding part projects in a thickness direction of the positioning tab. The arcuate locking portion is engaged with the positioning hole for positioning. The protruding part is in pressing contact with an inner wall of the positioning hole for positioning.

Optionally, in one embodiment of the present application, the insulator further includes a base and a cover mounted on the base. The base includes the first window and the second window. The cover includes the recess and defines a third window that is aligned with the second window and configured to communicate with the recess. The first electrical contact portion is configured to extend through the first window and is exposed at a top side of the base, and the second electrical contact portion is configured to extend through the second window and the third window and is exposed within the recess.

Optionally, in one embodiment of the present application, the base further includes a mounting groove communicating with the second window. A plurality of latching slots are arranged downward at a bottom portion of the mounting groove. Each of the plurality of latching slots includes a latching hook arranged on an inner wall. The cover includes a plurality of snap-fit tabs disposed at a lower side of the cover. Each of the plurality of snap-fit tabs is inserted into a corresponding one of the plurality of latching slots and engaged with the latching hook.

Optionally, in one embodiment of the present application, the base further defines an error proofing, a plurality of positioning grooves, and an error proofing positioning portion. The error proofing groove is arranged on an inner wall of the mounting groove. The plurality of positioning grooves is arranged on two sides of the base. The error proofing positioning portion protrudes outward and disposed on an outer side of the base.

The cover further includes a error proofing protrusion disposed on an outer side of the cover. The error proofing protrusion is configured to press into the error proofing groove.

To address the above technical problem, in a second aspect the present application provides a biosensor device. The biosensor device includes a board-end connector consistent with any one of embodiments mentioned foregoing, a housing, a circuit board and a sensor.

The circuit board and the sensor correspondingly serve as two electronic devices and are disposed within the housing. The board-end connector is disposed within the housing. The sensor is pressed from top to bottom into the recess of the board-end connector. A first conductive portion formed at a lower end of the sensor is in pressing contact with the second electrical contact portion of the second elastic arm. The circuit board is pressed from top to bottom to the board-end connector and the sensor. A third conductive portion of the circuit board is in pressing contact with the first electrical contact portion of the first elastic arm. A fourth conductive portion of the circuit board contacts a second conductive portion formed at an upper end of the sensor.

In one aspect, the present application provides a board-end connector including an insulator and a plurality of conductive members. Each of the plurality of conductive members includes a first elastic arm and a second elastic arm. The first elastic arm includes a first electrical contact portion. The second elastic arm includes a second electrical contact portion. The first elastic arm and the second elastic arm are electrically connected to each other and are correspondingly configured to electrically contact two electronic devices. A top surface of the first electrical contact portion is higher than a top surface of the second electrical contact portion.

By adopting the above technical solution, the present application provides the following beneficial effects over the prior art.
1. The first elastic arm and the second elastic arm of each of the plurality of conductive members are arranged on the same side, with the first electrical contact portion and the second electrical contact portion both exposed at the same side of the insulator and correspondingly configured to form elastic pressing contact with the two electronic devices to achieve electrical connection. The conductive members do not require soldering terminals and instead enable electrical connection through elastic pressing contact, allowing simplified assembly without being constrained by space limitations typically associated with soldering. Even when multiple conductive members are included, all first electrical contact portions or all second electrical contact portions can be simultaneously connected to a single electronic device in one operation, thereby simplifying and streamlining the assembly process. Additionally, one of the two electronic devices can be assembled within the recess of the insulator, the recess being configured to position the electronic device. This ensures stable elastic contact between the second electrical contact portion and the electronic device, facilitating proper alignment when the other electronic device is pressed onto the assembled electronic device and enabling stable electrical connection between the two electronic devices.
2. In the biosensor device, electrical connection between the board-end connector, the sensor, and the circuit board, such as PCB, is achieved through contact without soldering, thereby eliminating the soldering process and avoiding difficulties caused by space limitations. The first electrical contact portion and the second electrical contact portion, electrically connected to each other within the board-end connector, respectively form elastic pressing contact with the third conductive portion of the circuit board and the first conductive portion of the sensor. This configuration facilitates easy assembly and disassembly. Even when multiple conductive members are provided, the circuit board can simultaneously engage all of the first electrical contact portions, and the sensor can simultaneously engage all of the second electrical contact portions in a single operation, thereby simplifying operation and improving convenience.

### BRIEF DESCRIPTION OF DRAWINGS

FIG. 1 illustrates a schematic view of an internal structure of a portable blood glucose monitoring device in the prior art.

FIG. 2 is a perspective view of a board-end connector in accordance with an embodiment of the present application.

FIG. 3 is another perspective view of the board-end connector in accordance with the embodiment of the present application.

FIG. 4 is an exploded perspective view of the board-end connector in accordance with the embodiment of the present application.

FIG. 5 is a perspective view of a conductive member of the board-end connector in accordance with the embodiment of the present application.

FIG. 6 is another perspective view of the conductive member of the board-end connector in accordance with the embodiment of the present application.

FIG. 7 is a perspective view of a base of the board-end connector in accordance with the embodiment of the present application.

FIG. 8 is a perspective view of a cover of the board-end connector in accordance with the embodiment of the present application.

FIG. 9 is a perspective view of a sensor of a biosensor device in accordance with an embodiment of the present application.

FIG. 10 is an assembly view of the sensor and the board-end connector in the biosensor device in accordance with the embodiment of the present application.

FIG. 11 is an assembly view of the biosensor device in accordance with the embodiment of the present application.

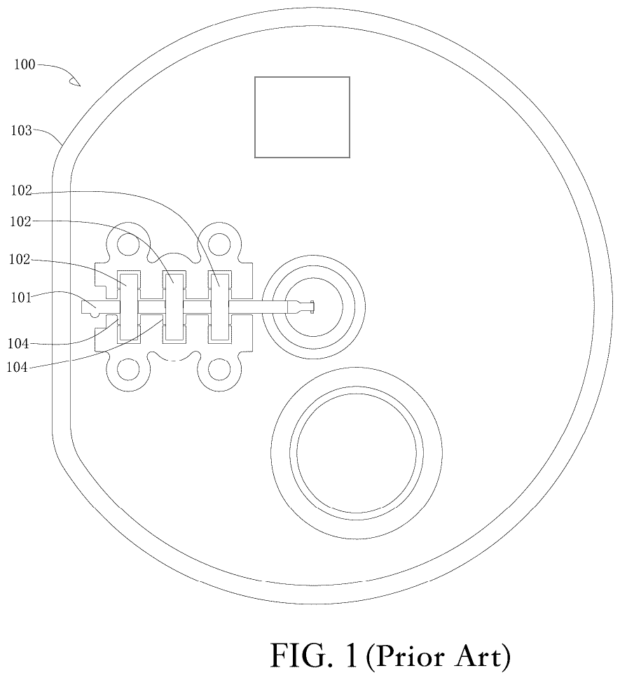

### DETAILED DESCRIPTION

Exemplary embodiments will now be described in detail with reference to the accompanying drawings. However, these embodiments can be implemented in various forms and should not be construed as limiting. Rather, they are provided to enhance the understanding of the present disclosure and to fully convey its concept to those skilled in the art. Furthermore, the specific embodiments described herein are for illustrative purposes only and do not limit the present application.

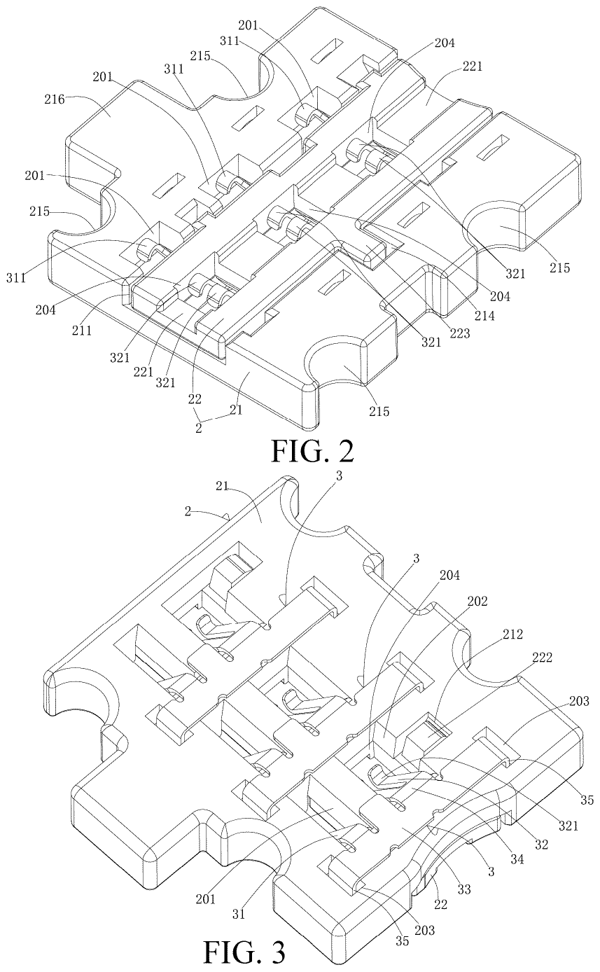

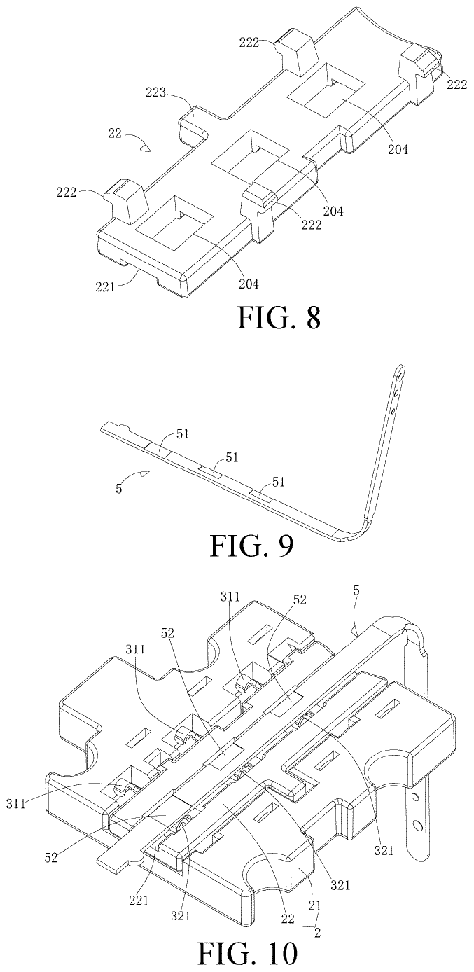

As shown in FIGS. 2 to 8, in one embodiment of the present application, a board-end connector is provided to electrically connect two electronic devices.

The board-end connector includes an insulator 2 and a plurality of conductive members 3 fixed to the insulator 2. The insulator 2 includes a recess 221 configured to receive or accommodate one of the two electronic devices. Preferably, the recess 221 may be a groove formed in the insulator 2. Each of the plurality of conductive member 3 includes a first elastic arm 31 and a second elastic arm 32 electrically connected to each other and correspondingly configured to electrically contact the two electronic devices. The first elastic arm 31 includes a first electrical contact portion 311 disposed at an upper end of the first elastic arm 31 and is exposed at an upper portion of the insulator 2. The second elastic arm 32 includes a second electrical contact portion 321 disposed at an upper end of the second elastic arm 32, and the second electrical contact portion 321 is exposed within the recess 221.

In an embodiment of present application, the first elastic arm 31 and the second elastic arm 32 of each conductive member 3 are arranged on the same side, such that the first electrical contact portion 311 and the second electrical contact portion 321 are both exposed on the same side of the insulator 2 and are correspondingly configured to elastically press contact the two electronic devices to establish electrical connection. The conductive member 3 achieves electrical connection with the two electronic devices by elastic pressing contact, without requiring soldering terminals for connection to either of the electronic devices. This approach simplifies assembly and avoids space limitations associated with soldering. Even when multiple conductive members 3 are provided, one of the electronic devices can be connected in one operation to all of the first electrical contact portions 311 or all of the second electrical contact portions 321, enabling simple and convenient assembly.

Additionally, after one of the two electronic devices is assembled in the recess 221 of the insulator 2, the recess 221 is configured to position the electronic device and ensure stable elastic pressing contact between the second electrical contact portion 321 of the second elastic arm 32 and the electronic device. This configuration prevents misalignment when the other electronic device is pressed against the assembled electronic device, thereby establishing a stable electrical connection between the two electronic devices.

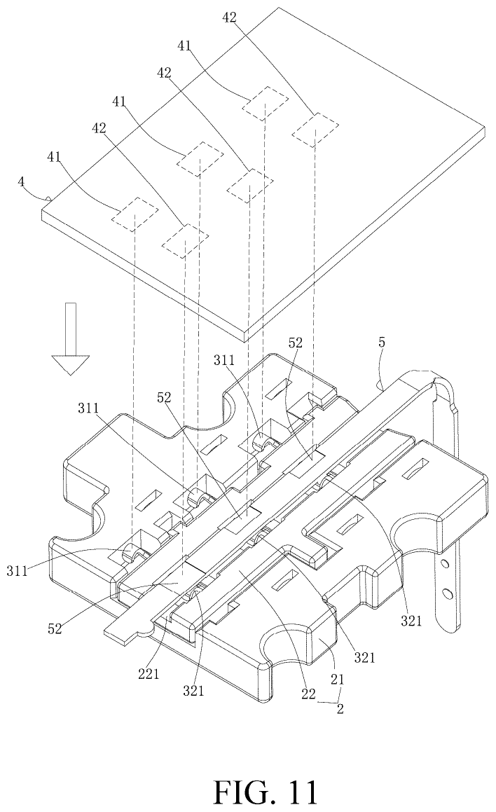

In this embodiment, referring to FIGS. 10 and 11, the two electronic devices refer to a circuit board 4, such as PCB, and a sensor 5. The sensor 5 is accommodated in the recess 221 of the insulator 2 and forms elastic pressing contact with the second electrical contact portion 321 to achieve electrical connection. The circuit board 4 is pressed onto the insulator 2 and the sensor 5 and forms elastic pressing contact with the first electrical contact portion 311 to achieve electrical connection. The circuit board 4 applies a pressure to the sensor 5, thereby enhancing the stability of the elastic pressing contact between the second electrical contact portion 321 of the second elastic arm 32 and the sensor 5, and improving the electrical connection. Optionally, the circuit board 4 and the sensor 5 may also be in electrical contact with each other, or they may remain electrically isolated, depending on specific application requirements.

The specific structure of each conductive member 3 is as follows:

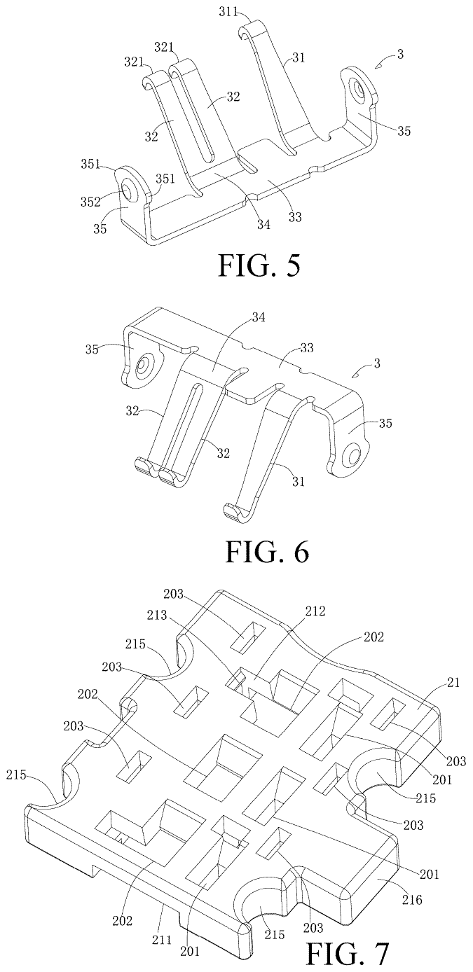

Referring to FIGS. 5 and 6, each conductive member 3 includes a main body portion 33, the first elastic arm 31, the second elastic arm 32, and two positioning tabs 35. The first elastic arm 31 and the second elastic arm 32 are bent and formed from a central region of the main body portion 33, and the two positioning tabs 35 are respectively bent and formed from both ends of the main body portion 33. The first elastic arm 31, the second elastic arm 32, and the two positioning tabs 35 are all exposed at an upper side of the main body portion 33, so that each conductive member 3 does not include any soldering terminal. That is, the first elastic arm 31 and the second elastic arm 32 are connected via the main body portion 33 to establish electrical connection.

The first electrical contact portion 311 is formed by bending the upper end of the first elastic arm 31. An upper surface of the first electrical contact portion 311 is an arcuate surface with a raised structure to facilitate electrical contact. Similarly, the second electrical contact portion 321 is formed by bending the upper end of the second elastic arm 32. An upper surface of the second electrical contact portion 321 is also an arcuate surface with a raised structure to facilitate electrical contact. In some embodiments, a top surface of the first electrical contact portion 311 is higher than a top surface of the second electrical contact portion 321. This arrangement allows the first electrical contact portion 311 to protrude beyond an upper portion of the insulator 2, while the second electrical contact portion 321 is exposed within the recess 221 (referring to FIG. 2). As a result, the second elastic arm 32 does not need to be longer than the first elastic arm 31.

To enhance the electrical connection performance, two second elastic arms 32 are provided and arranged in parallel. In other words, the two second elastic arms 32 are configured to establish electrical connection with the same position on the electronic device (i.e., the sensor 5), thereby improving connection reliability and stability while reducing the risk of misalignment that may cause connection failure. Structurally, root portions of the two second elastic arms 32 are connected to a bending portion 34, and the bending portion 34 is integrally connected to the main body portion 33, which increases the elastic deformability of the two second elastic arms 32 and further enhances the electrical connection performance between the conductive member 3 and the electronic device (i.e., the sensor 5).

The assembly configuration of the insulator 2 and the conductive member 3 includes at least the following two types

In a first assembly configuration, the insulator 2 is integrally fixed to the conductive members 3 by an insert molding process, providing simple assembly, high efficiency, and stable product structure.

In a second assembly configuration, the conductive member 3 is fixed to the insulator 2 by insertion, enabling convenient component replacement and reducing manufacturing cost.

In a preferred embodiment, the second assembly configuration described above is adopted.

As shown in FIGS. 2-4, the insulator 2 defines at least one first window 201 and at least one plurality of second window 202 arranged in a spaced manner, with the second window 202 extending through the recess 221, and the insulator 2 further defines at least one positioning hole 203. Each conductive member 3 includes at least one positioning tab 35, and the conductive member 3 is assembled to the insulator 2 by fitting and embedding the positioning tab 35 into a corresponding positioning hole 203.

In some embodiments, specifically, the insulator 2 defines a plurality of first windows 201 and a plurality of second windows 202 arranged in a spaced manner, with the second windows 202 extending through the recess 221, and the insulator 2 further defines a plurality of positioning holes 203. Each conductive member 3 includes a plurality of positioning tabs 35, and the conductive member 3 is assembled to the insulator 2 by fitting and embedding each positioning tab 35 into a corresponding one of the positioning holes 203. The first electrical contact portion 311 at the upper end of the first elastic arm 31 and the second electrical contact portion 321 at the upper end of the second elastic arm 32 correspondingly extend through a corresponding one of first window 201 and a corresponding one of second window 202. In other words, the first electrical contact portion 311 at the upper end of the first elastic arm 31 extends through the corresponding first window 201 and is exposed beyond an upper surface of the insulator 2, while the second electrical contact portion 321 at the upper end of the second elastic arm 32 extends through the corresponding second window 202 and into the recess 221.

In some embodiments, as shown in FIGS. 5-7, a specific assembly structure of one positioning tab 35 and one positioning hole 203 is described as follows. Both sides of an end portion of the positioning tab 35 extend along a width direction of the positioning tab 35 to form two arcuate locking portions 351. A protruding part 352 is formed at a central region of the end portion of the positioning tab 35 by a stamping process, projecting along a thickness direction of the positioning tab 35. The two arcuate locking portions 351 engage with the positioning hole 203 to achieve positioning in one direction, while the protrusion 352 presses against an inner wall of the positioning hole 203 to achieve tensioned positioning in another direction. In this way, a stable embedded and fixed assembly is formed between the positioning tab 35 and the positioning hole 203, allowing the conductive member 3 to be securely fitted to the insulator 2. Each arcuate locking portion 351 has an arcuate shape that facilitates smooth insertion into the positioning hole 203.

In some embodiments, the structure of the insulator 2 may be an integral structure, in which the first window 201, the second window 202, and the recess 221 are all disposed on the insulator 2.

In some embodiments, the structure of the insulator 2 may also be a split structure, the specific configuration being as follows:

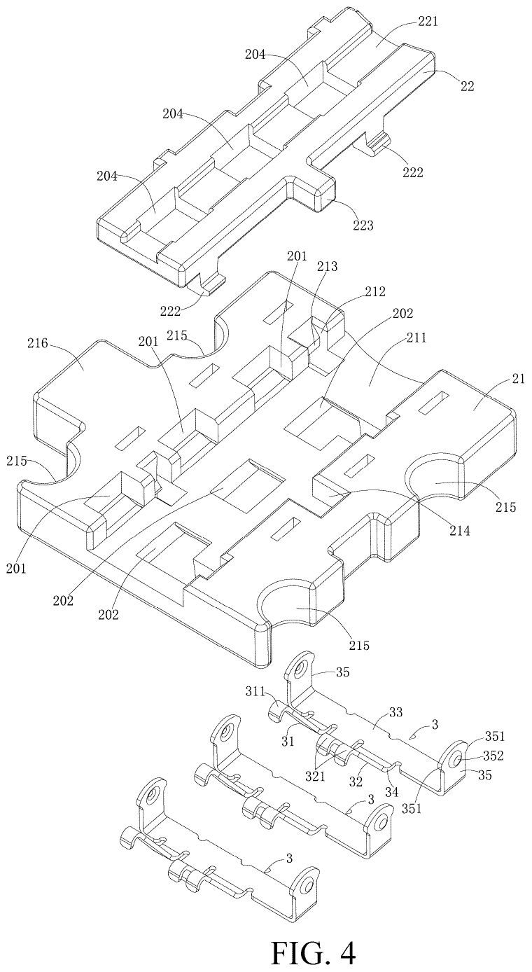

Referring to FIG. 4, the insulator 2 is a two-piece structure including a base 21 and a cover 22 fixedly mounted on the base 21, the base 21 and the cover 22 together forming an assembly configuration.

The base 21 is provided with the plurality of first windows 201 and the plurality of second windows 202, the cover 22 is provided with the recess 221, and the cover 22 further includes a third window 204 aligned with the second windows 202 and configured to communicate with the recess 221. The first electrical contact portion 311 extends through the first window 201 and is exposed at a top side of the base 21. The second electrical contact portion 321 extends through the second window 202 and the third window 204 and is exposed within the recess 221, that is, exposed within the cover 22. The cover 22 is configured to be assembled with one of the electronic devices, namely the sensor 5.

Since the cover 22 is detachably mountable relative to the base 21, a cover 22 with a recess 221 of suitable size can be selected according to the size of the electronic device (i.e., the sensor 5) and mounted on the base 21, thereby enabling use with electronic devices (i.e., the sensor 5) of different models or sizes.

The base 21 further includes a mounting groove 211 communicating with the second window 202s. A plurality of latching slots 212 are arranged at a bottom portion of the mounting groove 211, each latching slot 212 including a latching hook 213 arranged on an inner wall thereof. A plurality of snap-fit tabs 222 are disposed at a lower side of the cover 22. The cover 22 is fitted into the mounting groove 211, with each snap-fit tab 222 inserted into a corresponding latching slot 212 and engaged with the latching hook 213, thereby ensuring that the cover 22 is stably mounted to the base 21.

The base 21 further includes a error proofing groove 214 arranged on an inner wall of the mounting groove 211, and a error proofing protrusion 223 is disposed on an outer side of the cover 22. The error proofing protrusion 223 is pressed and fitted into the error proofing groove 214, thereby preventing the cover 22 from being reversely mounted relative to the base 21 during assembly, and achieving a error proofing effect. In addition, after the error proofing protrusion 223 is pressed into the error proofing groove 214, the error proofing protrusion 223 also serves as a limiting or positioning feature, thereby further enhancing the stability of the assembly between the cover 22 and the base 21.

The base 21 further includes a plurality of positioning grooves 215 arranged on two sides of the base 21, the positioning grooves 215 being configured to engage with positioning posts (not shown) inside a housing of a biosensor device so as to ensure that the board-end connector is stably mounted within the housing. In addition, the base 21 further includes a error proofing positioning portion 216 protruding outward from an outer side of the base 21, the error proofing positioning portion 216 being configured to prevent the board-end connector from being reversely mounted relative to the housing and thereby providing a reliable error proofing effect.

As described above, in the board-end connector of n, the first elastic arm 31 and the second elastic arm 32 of each conductive member 3 are disposed on the same side, with the first electrical contact portion 311 at the upper end of the first elastic arm 31 and the second electrical contact portion 321 at the upper end of the second elastic arm 32 both exposed at the same side of the insulator 2, correspondingly configured to elastically press against two electronic devices to establish. That is, the conductive member 3 of the present application does not require soldering terminals to be soldered to either of the two electronic devices, but instead establishes electrical connection with the two electronic devices through elastic pressing contact. This configuration enables simplified assembly without space limitations associated with soldering.

Even when multiple conductive members 3 are provided, one single electronic device can be simultaneously electrically connected in one operation to all first electrical contact portions 311 or all second electrical contact portions 321, thereby simplifying operation. In addition, after one of the two electronic devices is assembled or accommodated in the recess 221 of the insulator 2, the recess 221 positions the electronic device to ensure stable elastic pressing contact between the second electrical contact portion 321 of the second elastic arm 32 and the electronic device, thereby preventing misalignment when the other electronic device is pressed onto the assembled electronic device and enabling a stable electrical connection between the two electronic devices.

The present application further provides a biosensor device, which may be a continuous glucose monitor. Referring to FIGS. 2 to 11, the biosensor device includes the board-end connector described above, a housing (not shown), a circuit board 4 such as a printed circuit board (PCB), and a sensor 5. The circuit board 4 and the sensor 5 are disposed inside the housing and correspondingly serve as the two electronic devices. The board-end connector has already been clearly and fully described above, and thus is not described in further detail herein.

The board-end connector is fixed within the housing. The sensor 5 is pressed from top to bottom into the recess 221 of the board-end connector, with a first conductive portion 51 at a lower end of the sensor 5 pressed into contact with the second electrical contact portion 321 at the upper end of the second elastic arm 32. The circuit board 4 is pressed from top to bottom onto both the board-end connector and the sensor 5, with a third conductive portion 41 of the circuit board 4 pressed into contact with the first electrical contact portion 311 at the upper end of the second elastic arm 32, and a fourth conductive portion 42 of the circuit board 4 contacting a second conductive portion 52 (as shown in FIG. 11) formed at an upper end of the sensor 5, thereby establishing a series connection among the board-end connector, the sensor 5, and the circuit board 4.

In other words, electrical connection among the board-end connector, the sensor 5, and the circuit board 4 is achieved through contact without soldering, eliminating the soldering process and avoiding assembly difficulties caused by space limitations. In addition, the first electrical contact portion 311 and the second electrical contact portion 321, which are electrically connected within the board-end connector, are correspondingly pressed into elastic contact with the third conductive portion 41 at a lower end of the circuit board 4 and the first conductive portion 51 at the lower end of the sensor 5, thereby facilitating convenient assembly and disassembly. Even when multiple conductive members 3 are provided, the circuit board 4 can be simultaneously connected to all of the first electrical contact portions 311, and the sensor 5 can be simultaneously connected to all of the second electrical contact portions 321, both in a single operation, thereby simplifying and facilitating assembly.

It should be noted that the foregoing description is merely of specific embodiments of the present application and is not intended to limit the scope of implementation of the present application. Any equivalent variations or modifications made based on the structures, features, and principles described in the claims of the present application shall fall within the scope of the present application.

What is claimed is:

1. A board-end connector, comprising:
an insulator comprising a recess; and
a plurality of conductive members fixed on the insulator, each of the plurality of conductive members comprising:
a first elastic arm comprising a first electrical contact portion disposed at an upper end of the first elastic arm and exposed at an upper portion of the insulator; and
a second elastic arm comprising a second electrical contact portion disposed at an upper end of the second elastic arm and exposed in the recess;
wherein the first elastic arm and the second elastic arm are electrically connected to each other and are correspondingly configured to electrically contact two electronic devices;
wherein the recess is configured to accommodate one of the two electronic devices.

2. The board-end connector according to claim 1, wherein each of the plurality of conductive members further comprises a main body portion, the first elastic arm and the second elastic arm are connected via the main body portion, and a top surface of the first electrical contact portion is higher than a top surface of the second electrical contact portion.

3. The board-end connector according to claim 2, wherein each of the plurality of conductive members comprises two second elastic arms arranged in parallel and a bending portion, root portions of the two second elastic arms are connected to the bending portion, and the bending portion is integrally connected to the main body portion.

4. The board-end connector according to claim 1, wherein the insulator is integrally formed with and fixed to the plurality of conductive members by an insert molding process.

5. The board-end connector according to claim 1, wherein the plurality of conductive members are fixed to the insulator by an insertion;
wherein the insulator defines at least one first window, at least one second window and at least one positioning hole, and the first window and the second window are spaced apart from each other, such that the second window is configured to extend through the recess;
wherein each of the plurality of conductive members further comprises at least one positioning tab, and each of the plurality of conductive members is assembled to the insulator by fitting the positioning tab into the positioning hole, and the first electrical contact portion disposed at the upper end of the first elastic arm and the second electrical contact portion disposed at the upper end of the second elastic arm are configured to correspondingly extend through the first window and the second window.

6. The board-end connector according to claim 5, wherein the positioning tab comprises an arcuate locking portion and a protruding part, the arcuate locking portion is formed extending along a width direction on both sides of an end portion of the positioning tab, and the protruding part is formed by a stamping process at the end portion of the positioning tab and projects along a thickness direction of the positioning tab;
wherein the arcuate locking portion is engaged with the positioning hole for positioning, and the protruding part is in pressing contact with an inner wall of the positioning hole for positioning.

7. The board-end connector according to claim 5, wherein the insulator further comprises a base and a cover mounted on the base, the base comprises the first window and the second window, and the cover comprises the recess and defines a third window that is aligned with the second window and configured to communicate with the recess;
wherein the first electrical contact portion extends through the first window and is exposed on a top side of the base, and the second electrical contact portion extends through the second window and the third window and is exposed within the recess.

8. The board-end connector according to claim 7, wherein the base further comprises a mounting groove communicating with the second window, a plurality of latching slots are arranged downward at a bottom portion of the mounting groove, and each of the plurality of latching slots comprises a latching hook arranged on an inner wall;
wherein the cover comprises a plurality of snap-fit tabs disposed at a lower side of the cover, and each of the plurality of snap-fit tabs is inserted into a corresponding one of the plurality of latching slots and engaged with the latching hook.

9. The board-end connector according to claim 8, wherein the base further defines an error proofing groove, a plurality of positioning grooves and an error proofing positioning portion, the error proofing groove is arranged on an inner wall of the mounting groove, the plurality of positioning grooves is arranged on two sides of the base, and the error proofing positioning portion protrudes outward and disposed on an outer side of the base;
wherein the cover further comprises an error proofing protrusion disposed on an outer side of the cover, and the error proofing protrusion is configured to press into the error proofing groove.

10. A biosensor device, comprising:
a board-end connector recited in claim 1;
a housing;
a circuit board; and
a sensor;
wherein the circuit board and the sensor correspondingly serve as two electronic devices and are disposed within the housing;
wherein the board-end connector is disposed within the housing, the sensor is pressed from top to bottom into the recess of the board-end connector, a first conductive portion formed at a lower end of the sensor is in pressing contact with the second electrical contact portion of the second elastic arm;
wherein the circuit board is pressed from top to bottom to the board-end connector and the sensor, a third conductive portion of the circuit board is in pressing contact with the first electrical contact portion of the first elastic arm, and a fourth conductive portion of the circuit board contacts a second conductive portion formed at an upper end of the sensor.

11. The biosensor device according to claim 10, wherein the plurality of conductive members are fixed to the insulator by an insertion;
wherein the insulator defines at least one first window, at least one second window and at least one positioning hole, and the first window and the second window are spaced apart from each other, such that the second window is configured to extend through the recess;
wherein each of the plurality of conductive members further comprises at least one positioning tab, and each of the plurality of conductive members is assembled to the insulator by fitting the positioning tab into the positioning hole, and the first electrical contact portion disposed at the upper end of the first elastic arm and the second electrical contact portion disposed at the upper end of the second elastic arm are configured to correspondingly extend through the first window and the second window.

12. The biosensor device according to claim 11, wherein the positioning tab comprises an arcuate locking portion and a protruding part, the arcuate locking portion is formed extending along a width direction on both sides of an end portion of the positioning tab, and the protruding part is formed by a stamping process at the end portion of the positioning tab and projects along a thickness direction of the positioning tab;
wherein the arcuate locking portion is engaged with the positioning hole for positioning, and the protruding part is in pressing contact with an inner wall of the positioning hole for positioning.

13. The biosensor device according to claim 11, wherein the insulator further comprises a base and a cover mounted on the base, the base comprises the first window and the second window, and the cover comprises the recess and defines a third window that is aligned with the second window and configured to communicate with the recess;
wherein the first electrical contact portion extends through the first window and is exposed on a top side of the base, and the second electrical contact portion extends through the second window and the third window and is exposed within the recess.

14. The biosensor device according to claim 13, wherein the base further comprises a mounting groove communicating with the second window, a plurality of latching slots are arranged downward at a bottom portion of the mounting groove, and each of the plurality of latching slots comprises a latching hook arranged on an inner wall;
wherein the cover comprises a plurality of snap-fit tabs disposed at a lower side of the cover, and each of the plurality of snap-fit tabs is inserted into a corresponding one of the plurality of latching slots and engaged with the latching hook.

15. The biosensor device according to claim 14, wherein the base further defines an error proofing groove, a plurality of positioning grooves and an error proofing positioning portion, the error proofing groove is arranged on an inner wall of the mounting groove, the plurality of positioning grooves is arranged on two sides of the base, and the error proofing positioning portion protrudes outward and disposed on an outer side of the base;
wherein the cover further comprises an error proofing protrusion disposed on an outer side of the cover, the error proofing protrusion is configured to press into the error proofing groove.

16. A board-end connector, comprising:
an insulator; and
a plurality of conductive members fixed on the insulator, each of the plurality of conductive members comprising:
a first elastic arm comprising a first electrical contact portion; and
a second elastic arm comprising a second electrical contact portion;
wherein the first elastic arm and the second elastic arm are electrically connected to each other and are correspondingly configured to electrically contact two electronic devices;
wherein a top surface of the first electrical contact portion is higher than a top surface of the second electrical contact portion.

17. The board-end connector according to claim 16, wherein the insulator comprises a recess configured to accommodate one of the two electronic devices.

18. The board-end connector according to claim 17, wherein each of the plurality of conductive members further comprises a main body portion, the first elastic arm and the second elastic arm are connected via the main body portion, and the first electrical contact portion and the second electrical contact portion are correspondingly configured for elastic pressing contact with the two electronic devices to establish electrical connections.

19. The board-end connector according to claim 18, wherein the insulator defines at least one first window, at least one second window and at least one positioning hole, the first window and the second window are spaced apart from each other, the second window is configured to extend through the recess;
wherein each of the plurality of conductive members further includes at least one positioning tab, and each of the plurality of conductive members is assembled to the insulator by fitting the positioning tab into the positioning hole, the first electrical contact portion disposed at the upper end of the first elastic arm and the second electrical contact portion disposed at the upper end of the second elastic arm are configured to correspondingly extend through the first window and the second window.
